(19) United States
(12) Patent Application Publication
(10) Pub. No.: US 2026/0334994 A1
(43) Pub. Date: Oct. 29, 2026

(54) AN ELECTROPORATION PROBE AND APPARATUS

(71) Applicant: MIRAI MEDICAL LIMITED, Galway (IE)

(72) Inventors: Declan SODEN, Galway (IE); Colin FORDE, Galway (IE); Jason McNAMARA, Galway (IE); Cian KERRIGAN, Galway (IE)

(73) Assignee: MIRAI MEDICAL LIMITED, Galway (IE)

(21) Appl. No.: 19/101,675

(22) PCT Filed: Sep. 1, 2023
(86) PCT No.: PCT/EP2023/073999

§ 371 Date: Feb. 6, 2025

(30) Foreign Application Priority Data
Sep. 2, 2022 (EP) 22193699.0

(51) Int. Cl. A61B 18/00; A61B 1/018; A61B 17/00

(52) U.S. Cl. CPC A61B 18/00; A61B 1/018; A61B 2017/00566; A61B 2018/0016; A61B 2018/00178; A61B 2018/00184; A61B 2018/00214; A61B 2018/00613; A61B 2018/00744; A61B 2018/00767; A61B 2018/00982

(57) ABSTRACT

An electroporation probe has an elongate support with conductors linked to an inner electrode and an outer electrode to engage tissue. The inner electrode may be in the form of a needle to penetrate tissue on-axis, or it may just push against tissue. The outer electrode engages tissue radially outwardly of the inner electrode, with a pressing action. A large volume of tissue can be treated, in a manner which is very accurately located, in a conical pattern with an apex at the distal tip of the inner electrode and expanding proximally to the outer electrode.

### INTRODUCTION

The present invention relates to electroporation treatment, also known as Pulsed Electric Field (“PEF”) treatment.

Such treatment is for treating/ablating tissue or tumours within body structures such as the oesophagus, stomach, colon and other Gastrointestinal areas particularly but the human body more generally including cutaneous disease.

Known electroporation probes can sometimes be less effective than desired as it may be difficult or impossible to reach the target site and adequately engage the tissue (such as a tumour), and also tissue location can be at an angle to the direction of movement of the endoscope, for example, at a junction such as Gastro-oesophageal junction, or in a location that requires retroflexion of the endoscope, thus proving difficult to reach the target site and adequately engage the tumour. In addition, existing electroporation probes for use endoscopically are large in profile and thus reduce the ability of the endoscope to navigate successfully through more challenging anatomies as a result, a probe which can be delivered through typical endoscopic tools instrument channel without in any manner effecting the endoscopes navigability would be advantageous. Also, tumours can be large and often embedded deep in the wall of the anatomical structure where the depth of electric field with current modalities cannot reach. Also, the area to be treated can be inconsistent and non-uniform and cover a large area which requires a large number of applications of current treatment modalities. Also, visualisation of the tumour may be difficult due to the location of the tumour within the anatomy.

The present invention is directed towards providing an improved electroporation probe and apparatus to address these problems.

### SUMMARY OF THE INVENTION

We describe an electroporation probe comprising:
an elongate support with conductors, said elongate support supporting:
   an inner electrode linked with at least one of said conductors and being configured to engage tissue, and
   an outer electrode linked with at least one of said conductors and being configured to engage tissue radially outwardly and proximally of the inner electrode.

In some preferred examples, the inner electrode is configured to penetrate tissue. In some preferred examples, the inner electrode comprises a needle. In some preferred examples, the inner electrode is mounted along a longitudinal axis of the elongate support.

In some preferred examples, the outer electrode is configured to contact tissue over an annular area around the axis of the inner electrode, and proximally of the distal end of the inner electrode. In some preferred examples, the elongate support comprises an endoscope, and the electrodes are mounded in a collapsed state within a working or biopsy channel of the endoscope.

In some preferred examples, the inner electrode is hollow for at least some of its length and is configured for delivery of a fluid to tissue being treated. In some preferred examples, the needle has radial apertures for radial delivery of a fluid in addition to or instead of through a needle tip.

In some preferred examples, the outer electrode is deformable for engagement with a proximal facing surface of tissue being treated. In some preferred examples, the outer electrode comprises an expanding body supporting or being integral with electrode elements.

In some preferred examples, the outer electrode comprises an expanding mesh. In some preferred examples, the outer electrode comprises non-meshing wire elements.

In some preferred examples, the outer electrode comprises an expanding body supporting discrete electrode elements at a distal end thereof. In some preferred examples, the elongate support comprises an endoscope which is configured for being in retroflexion or any position between straight and full retroflexion.

In some preferred examples, the needle depth is adjustable relative to the elongate support. In some preferred examples, the outer electrode diameter/lateral dimension is adjustable.

In some preferred examples, the elongate support is capable of being steered and/or positioned with respect to the outer electrode and the endoscope. In some preferred examples, the outer and/or inner electrodes are steerable in relation to the elongate support.

In some preferred examples, the inner electrode is steerable either with the elongate support or relative to the elongate support.

In some preferred examples, the inner electrode includes an electrode element which is not needle-shaped but has a lateral dimension in the range of 1 mm to 10 mm to press distally against tissue being treated. In some preferred examples, the inner electrode comprises an expanding mesh.

In some preferred examples, the inner electrode is deformable for engagement with a proximal facing surface of tissue being treated. In some preferred examples, the inner electrode comprises an expanding body supporting electrode elements. In some preferred examples, the inner electrode comprises non-meshing wire elements; and the inner electrode may comprise an expanding body supporting discrete electrode elements at a distal end thereof.

In some preferred examples, the inner electrode diameter/lateral dimension is adjustable. In some preferred examples, the outer electrode includes a suction element part to create a vacuum between the outer electrode and target tissue.

In some preferred examples, the inner electrode includes an element for gripping tissue.

In some preferred examples, the inner electrode includes an expandable body having a maximum lateral dimension less than that of the outer electrode.

In some preferred examples, the inner electrode comprises wire loops which do not connect to each other when relaxed.

In some preferred examples, in a probe relaxed and unconstrained state, the inner electrode has a lateral dimension which is 1% to 80% of the lateral dimension of the outer electrode, more preferably 20% to 50% of the lateral dimension of the outer electrode.

In some preferred examples, the inner electrode and the outer electrode are collapsed in the elongate support and are expandable when the elongate support is retracted.

We also describe an electroporation apparatus comprising a probe of any example described herein and a pulse generator linked with said conductors for delivery of pulses to the electrodes.

In some preferred examples, the apparatus further comprises a pump for delivery of a fluid through the inner electrode.

In some preferred examples, the pulse generator is configured to operate the outer electrode as a dispersive pad.

In some preferred examples, the method comprising deploying a probe of an apparatus of any example within the body so that the inner electrode and the outer electrode contact tissue with the outer electrode radially outside of the inner electrode and spaced apart from the inner electrode and applying voltage pulses to the electrodes for electroporation treatment of the tissue between and adjacent said electrodes.

In some preferred examples, the inner electrode is moved longitudinally to provide a desired action of pressing against or penetrating the tissue.

In some preferred examples, the outer electrode is operated as a dispersive pad.

In some preferred examples, the inner electrode is moved longitudinally relative to the outer electrode to achieve a desired differential in longitudinal positions of said electrodes.

### ADDITIONAL STATEMENTS

We also describe an electroporation probe comprising an elongate support with conductors, an inner electrode linked with a conductor and being configured to engage tissue, and an outer electrode linked with a conductor and being configured to engage tissue radially outwardly of the inner electrode.

In some examples, the inner electrode is configured to penetrate tissue. In some examples, the inner electrode comprises a needle. In some examples, the inner electrode is mounted as an extension of the elongate support. In some examples, the elongate support comprises an endoscope. In some examples, the inner electrode is hollow for at least some of its length and is configured for delivery of a fluid to tissue being treated. In some examples, the needle has radial apertures for radial delivery of a fluid in addition to or instead of through a needle tip.

In some examples, the outer electrode is deformable for engagement with a proximal facing surface of tissue being treated. In some examples, the outer electrode comprises an expanding body supporting or being integral with electrode elements. In some examples, the outer electrode comprises an expanding mesh. In some examples, the outer electrode comprises non-meshing wire elements. In some examples, the outer electrode comprises an expanding body supporting discrete electrode elements at a distal end thereof, and optionally the elements are disc shaped.

In some examples, the inner and outer electrodes are capable of being deployed and retrieved even when the elongate support endoscope is in retroflexion or any position between straight and full retroflexion. In some examples, the inner electrode needle depth is adjustable relative to the elongate support.

In some examples, the outer electrode diameter/lateral dimension is adjustable. In some examples, the elongate support is capable of being steered/positioned with respect to the outer electrode and the endoscope. In some examples, the outer and/or inner electrodes are steerable in relation to the elongate support. In some examples, the inner electrode is steerable either with the elongate support or relative to the elongate support. In some examples, the inner electrode includes an electrode element which is not needle-shaped but has a lateral dimension in the range of 1 mm to 10 mm to press distally against tissue being treated.

We also describe an electroporation apparatus comprising a probe of any example described herein and a pulse generator linked with said conductor for delivery of pulses to the electrodes. In some examples, the apparatus further comprises a pump for delivery of a fluid through the inner electrode.

### DETAILED DESCRIPTION OF THE INVENTION

The invention will be more clearly understood from the following description of some embodiments thereof, given by way of example only with reference to the accompanying drawings in which:

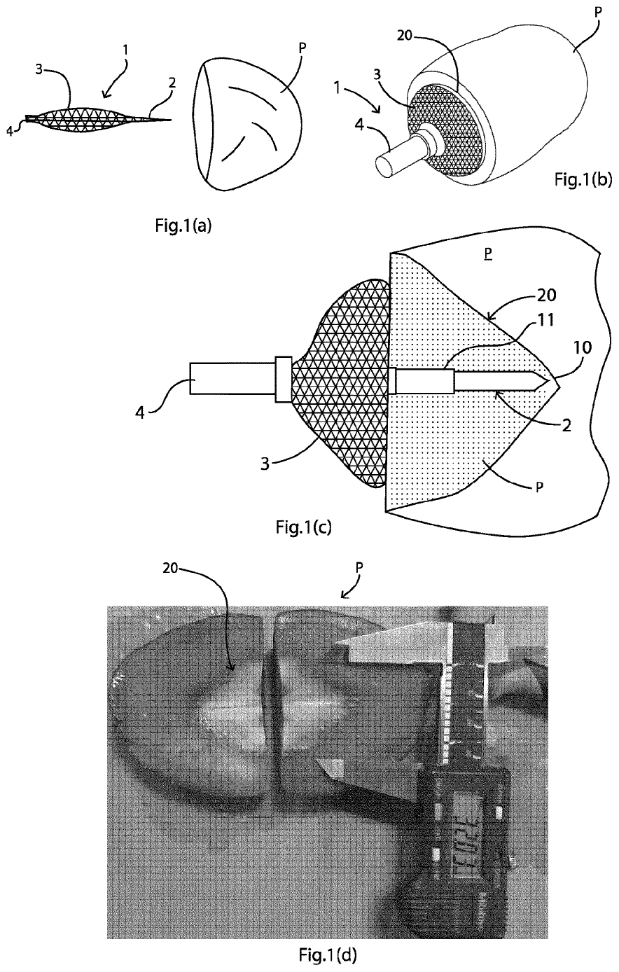

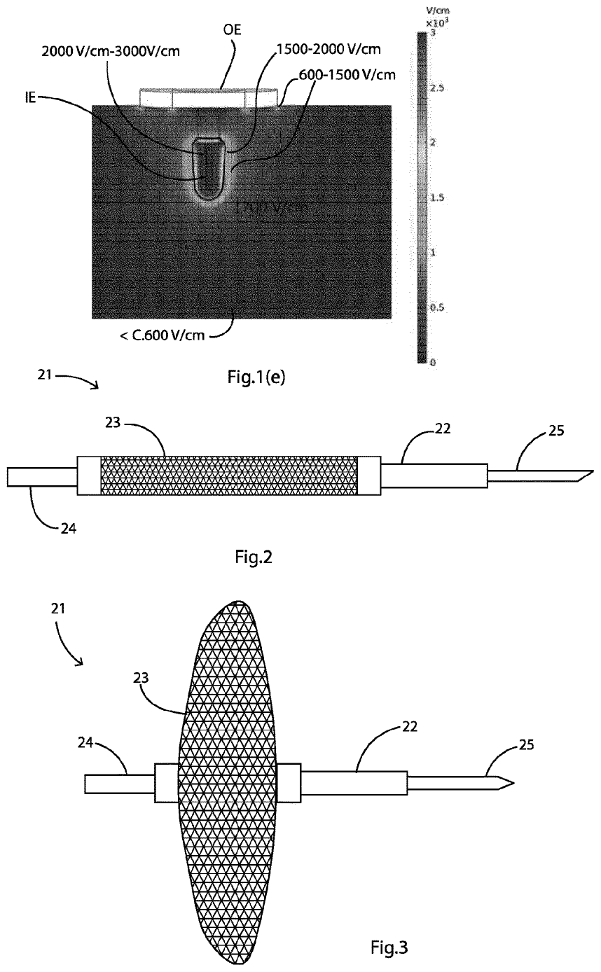

FIGS. 1(a), 1(b), and 1(c) are views showing use of an electroporation probe of the invention, having an expanding body made from an interwoven metal mesh, the use being in a potato for test purposes, this having a broadly similar set of characteristics to tissue to be treated, and in which FIG. 1(c) in particular illustrates the treated volume; FIG. 1(d) is an image showing a section through a treated region of a potato in a test; and FIG. 1(e) is a computer generated simulation image illustrating applied voltage patterns for an probe with an outer electrode OE and an inner electrode IE;

FIG. 2 shows another probe before deployment, and FIG. 3 shows it with compression of the expanding body to illustrate the shape of the expanding body when not compressed on the distal side as it would be in use;

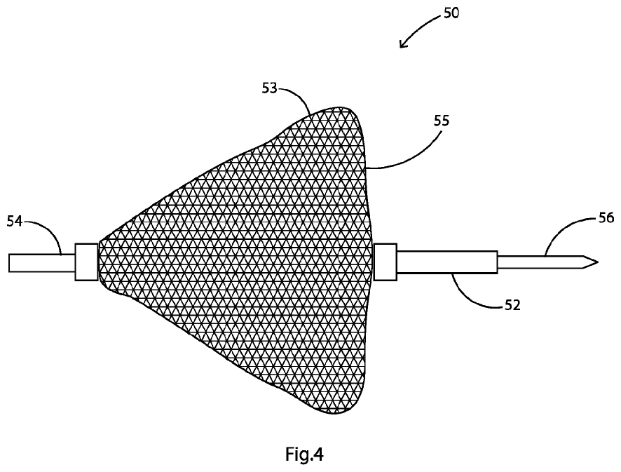

FIG. 4 shows an expanding body variant which has a shape with a distal part which is close to radial, to confer better contact between the expanding body and the tissue against which it is to be pushed;

FIG. 5 shows a different probe, in which there is not an interwoven mesh but instead a series of shape memory alloy wire loops which are arranged circumferentially similar to the petals of a flower, and which are heat-set to have the shape of an open flower when released from an overlayed sheath;

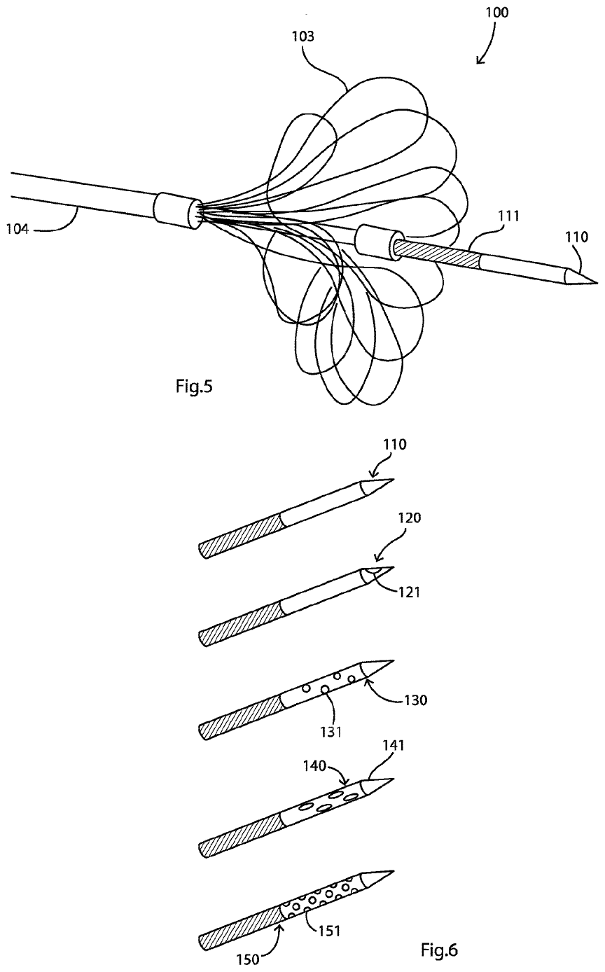

FIG. 6 is a set of perspective views showing alternative needle configurations for the inner electrode, some with radial pores/slots for flow of a treatment substance such as foam;

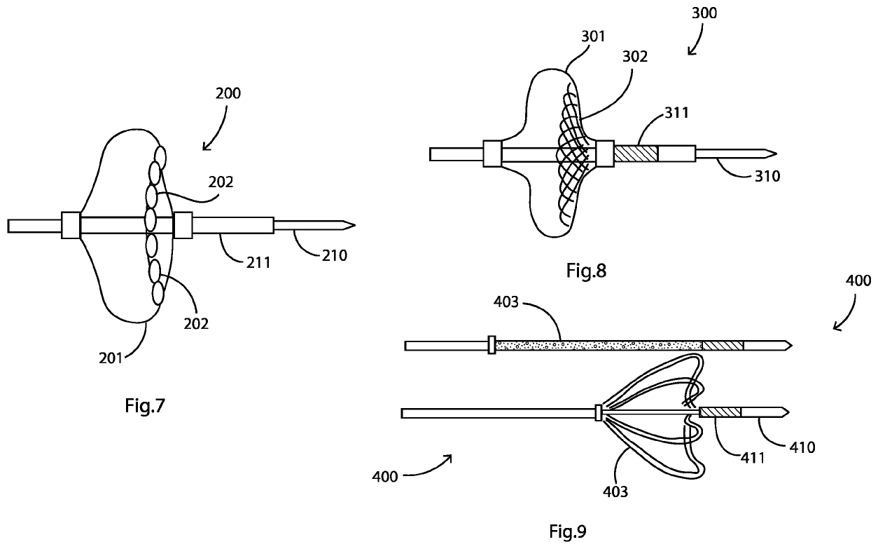

FIG. 7 is a diagrammatic side view of an alternative probe, in this case with disc-shaped electrode elements on the outer electrode, particularly suited for optimum contact with the proximal-facing surface of tissue being treated;

FIG. 8 is a diagrammatic side view of a further alternative probe, in this case with a mesh of electrode wires on the distal end of the mesh-shaped outer electrode or balloon;

FIG. 9 is a diagrammatic side view of a further alternative probe, in this case with circumferentially arranged non-meshing wires which are configured to extend radially inwardly and proximally at the distal end;

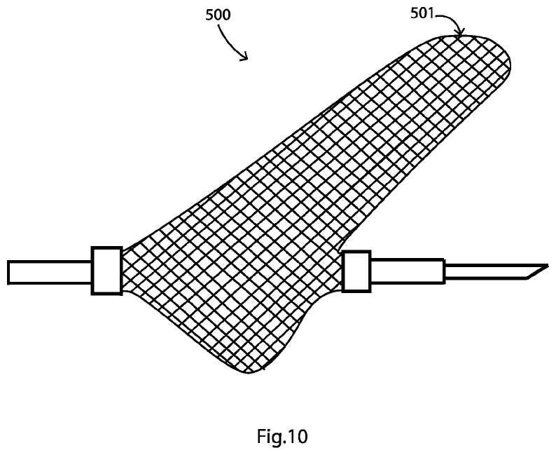

FIG. 10 is a diagrammatic side view of an alternative probe, in this case with an off-centre, asymmetric, expanding body which may be better suited for full tissue engagement when probes of this type are inserted into tissue at an angle due to geometric obstructions which would prevent their use perpendicular to the tissue to be treated;

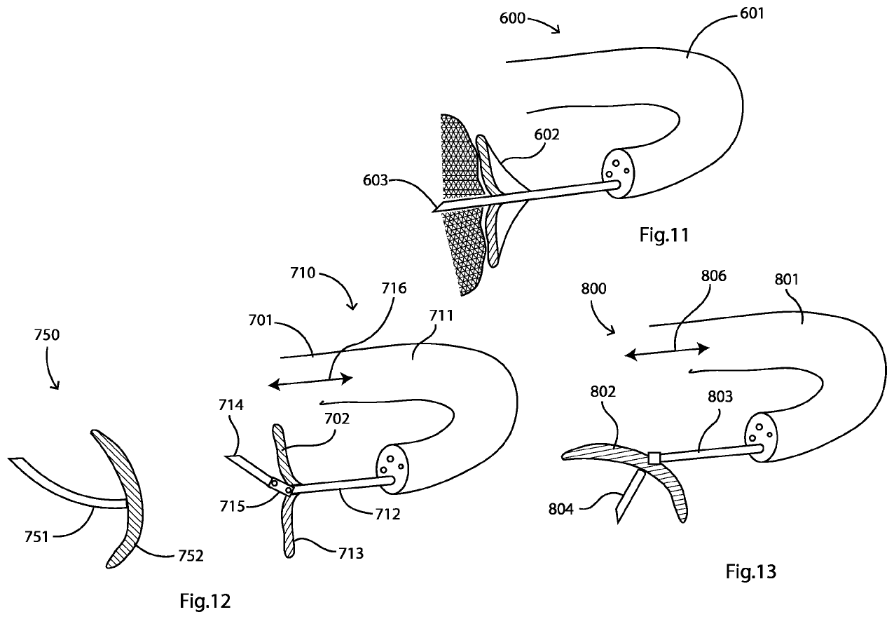

FIGS. 11, 12, and 13 are diagrammatic perspective views of alternative probes with directionality in articulation of the support rod and/or the needle electrode; and

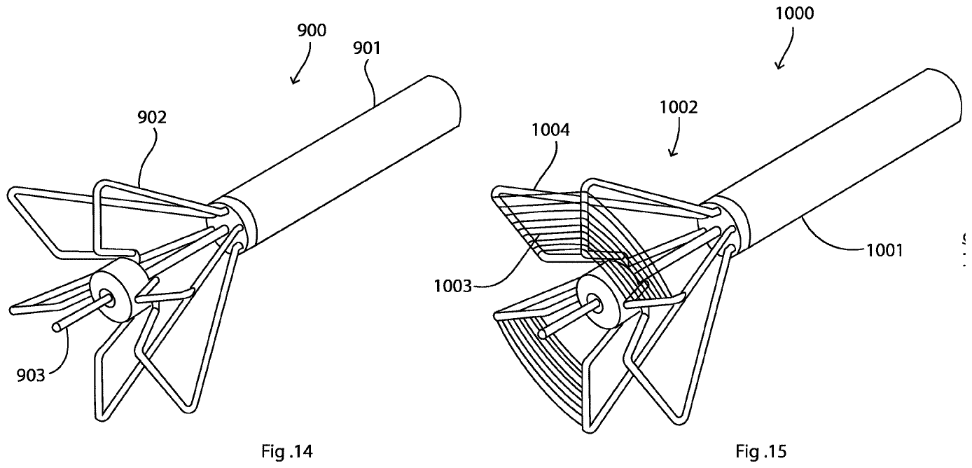

FIGS. 14 and 15 are views of alternative probes with different arrangements of non-meshing conductors,

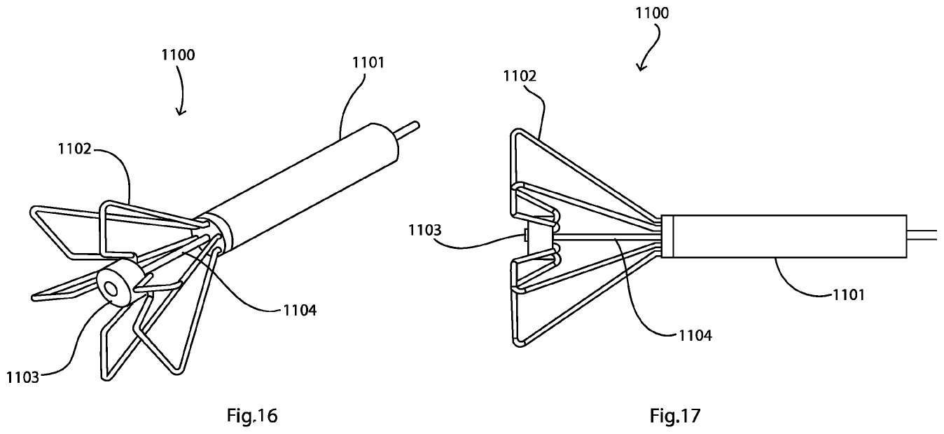

FIGS. 16 and 17 are views of alternative probes having an inner electrode which is not configured to penetrate tissue, but rather to push against it,

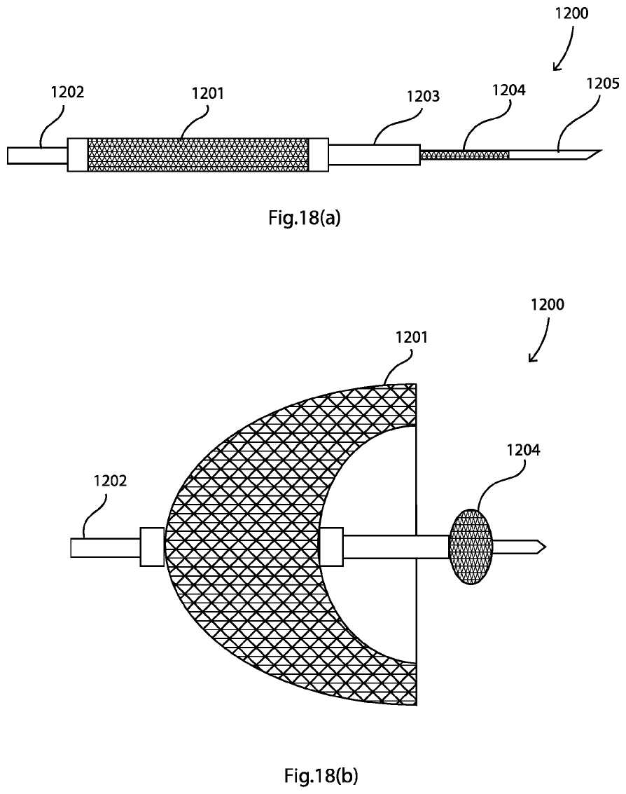

FIGS. 18 (a) and 18 (b) illustrate two states of a probe of the invention, wherein the inner electrode is expandable and can expand to a lesser radial extent than the other electrode,

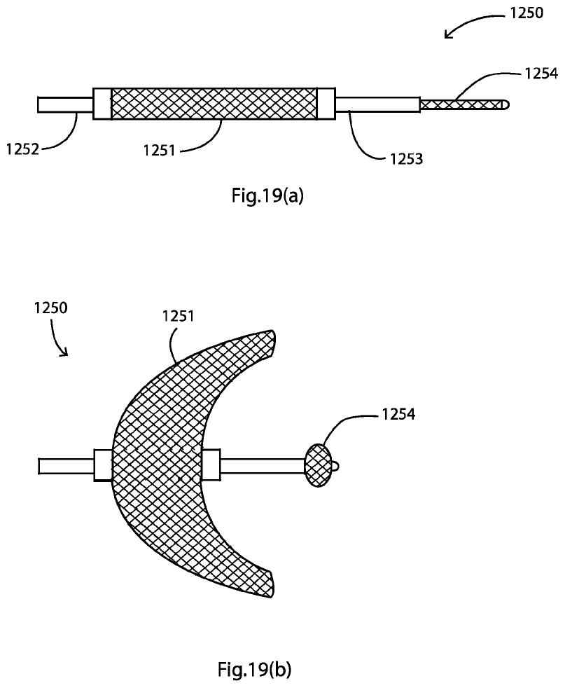

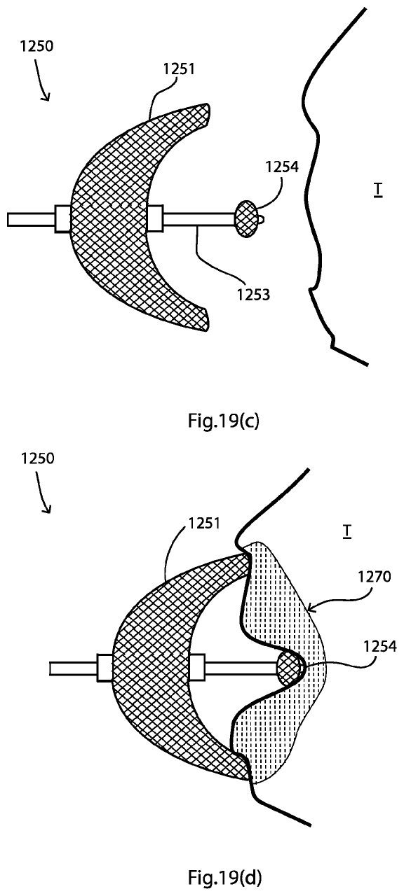

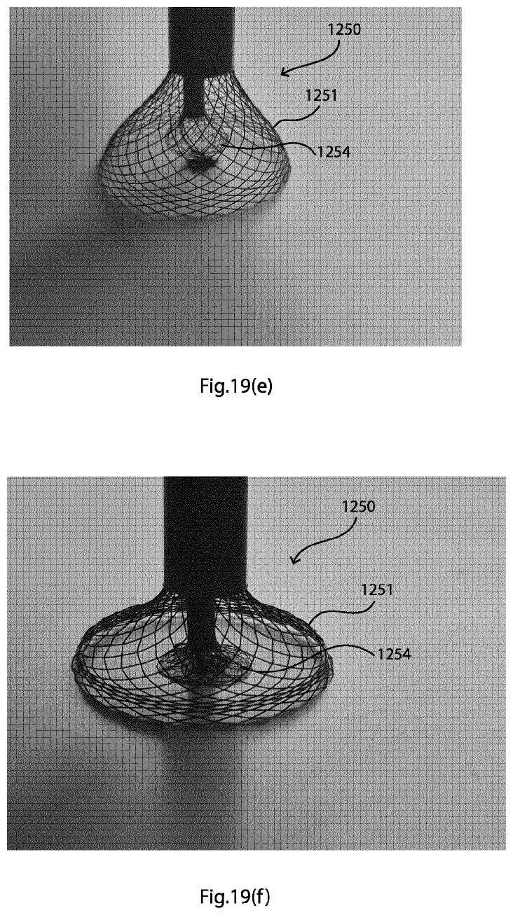

FIGS. 19(a) to (f) inclusive show an alternative probe in which the inner electrode comprises an expanding mesh which is not configured to penetrate tissue, and in particular:
FIG. 19(a) is a side view when the probe is collapsed prior to deployment,
FIG. 19(b) is a side view with the probe expanded,
FIG. 19(c) is a diagrammatic side view showing the probe approaching a tissue surface,
FIG. 19(d) is a diagrammatic side view showing the probe with the electrodes in contact with a tissue surface, showing how both of the electrodes push against the tissue surface to indent it rather than penetrate it and the region of treatment is illustrated,
FIG. 19(e) is an image of a prototype of the probe shown in FIGS. 19(a) to (d), and
FIG. 19(f) is an image showing this prototype when pressed against a hard surface to illustrate how it deforms upon contact with tissue.

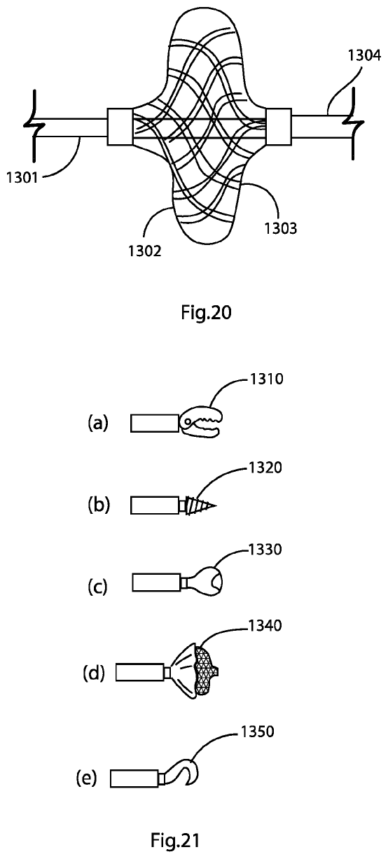

FIG. 20 illustrates a probe without an inner electrode, and FIGS. 21(a) to (e) inclusive show options for inner electrodes which may be provided, which facilitate secure temporary attachment of the probe to tissue.

### DETAILED DESCRIPTION OF THE INVENTION

various embodiments, an electroporation probe of the invention allows physicians to treat a variety of shapes of tumours within the body with an ability to:
consistently successfully access the target,
see the target lesion during or just before treatment,
have good capability to manoeuvre,
control the depth of treatment, and
control the width of treatment.

In examples of the invention, an electroporation probe has an elongate support with conductors linked to an inner electrode and an outer electrode to engage tissue. The inner electrode may be in the form of a needle to penetrate tissue on-axis, or it may just push against tissue. The outer electrode engages tissue radially outwardly of the inner electrode, with a pressing action. A large volume of tissue can be treated, in a manner which is very accurately located, in a conical pattern with an apex at the distal tip of the inner electrode (2 in FIGS. 1(a) to 1(c)) and expanding proximally to the outer electrode (3 in FIGS. 1(a) to 1(c)).

Referring to FIG. 1 an electroporation probe 1 comprises an inner electrode provided by a leading needle 2, proximally of which there is an outer electrode provided by an expanding basket 3 with an exposed wire mesh forming an outer electrode, all mounted to a flexible rod 4. A pulse generator, not shown, is linked to drive the inner and outer electrodes 2 and 3, and there is an insulating sheath 11 around the rod 4 between the outer electrode 3 and the inner electrode 2. The pulse generator linked to the conductors at the proximal end of the rod 4 may be of any suitable type, and an example is described in our published specification WO2021/043779 (Mirai Medical Ltd.), the contents of which are incorporated by reference.

In use, as shown in FIGS. 1(a) to (e), the probe 1 is manoeuvred to the target site, and the user operates an actuator to unsheathe the needle 2 based on the depth of treatment required. This accounts for the shrouded portion between the needle electrode 2 and the expanding electrode 3 which will also contact tissue. The user then advances the probe with the needle 2 penetrating the tissue to the user-defined depth. The user then actuates the expanding electrode body to its expanded state. The outer electrode 3 is configured to contact tissue over an annular area around the axis of the inner electrode 2 and of the elongate support 4, and proximally of the distal end of the inner electrode. When satisfied with positioning, the user interacts with the pulse generator to control pulses delivered to the needle 2 and to the mesh metal basket 3 (or nonmetal but with electrodes on its frame or body) pushed against the tissue front surface. There are separate conductors contained within the sheath 4, one for each of the needle 2 (inner electrode) and the basket 3 (outer electrode). Control of the needle 2 penetration depth allows the physician to treat lesions of a location, depth and spread they would not have been able to before. As there is just one needle to control and thus position the invention provides major advantages in utility, safety and speed over multi needle device designs. In addition, the one needle may be more robust than those that likely would be chosen in a multi needle arrangement. The outer electrode 3 allows the desired current carrying capacity and is able to conform to the shape of the surface of the tissue facing proximally.

In various examples the leading needle could be solid, hollow and/or tipped in such a manner as to allow delivery of chemotherapy or other treatment fluid to cause cell death or manage tissue impedance. The fact that the needle can be both the centre (inner) electrode and also a conduit for delivery of the required substance means the substance is always in the correct location for the treatment, which may maximise efficacy. The requirement not to use a secondary device for substance delivery potentially decreases procedural time and risk while enhancing procedure safety and accuracy. In delivery of an Electrochemotherapy (ECT) treatment the procedure outcome is likely to be more efficacious due to the elimination of a time delay between intra-tumoural injection and subsequent electroporation as it is recommended that this take place as fast as possible as the injected agent will have less time to dissipate. Where the needle is hollow the elongate support includes a conduit for flow of fluid to the needle. This conduit runs parallel to the insulated power conductors delivering the pulses to the electrodes.

In some examples, the probe may include a removable sheath for the expanding body. Such an expanding body may be unsheathed from the proximal end in a manner well known in the art of balloons and stents; this would allow the physician to decide, for example, to unsheathe enough basket material such that the resulting basket has a certain diameter such as 20 mm: the amount unsheathed defining the basket resulting diameter and thus the spread of the electroporation experienced by the tissue. The region 20 is generally conical, with an apex at the distal end (tip) of the inner electrode 2 and expanding proximally.

As shown in FIGS. 1(c) and 1(d) the probe produces an Irreversibly Electroporated region that demonstrates the electric field produced by this device is cone shaped, more specifically “Strawberry shaped”. Deployment of the central electrode to a shallower depth produces a field shape that could be described as ellipsoid. The region demonstrating cell wall disturbance for the test shown is illustrated in FIGS. 1(c) and 1(d) and is identified by the numeral 20. FIG. 1(d) demonstrates the irreversibly electroporated region generated in a potato using a prototype device to deliver eight 600 V unipolar square wave pulses at a rate of 10 Hz, the affected region in this example was estimated at 10 cubic centimetres however this is dependent on the tissue, the pulse type and quantity and the specific prototype dimensions but serves to demonstrate that relatively large volumes of tissue can be treated using a probe of the invention.

FIG. 1(e) is a computer-generated simulation image illustrating applied voltage patterns for a probe with an outer electrode OE and an inner electrode IE. In this simulation the outer electrode OE has a washer-shaped configuration, and the inner electrode IE has a needle configuration. In this simulation the electroporation field ranges from a value in the range of 2 kV/cm to 3 k V/cm around the inner electrode IE to a value in the range of 600 kV/cm to 1500 kV/cm close to the outer electrode OE. These parameters show that the probe may be used in surgery in which the outer electrode acts as a dispersive pad.

The probe 1 preferably has a maximum diameter of less than 11 mm (a common endoscope diameter). It more preferably has a maximum diameter of less that 3.7 mm, which is a common endoscope instrument channel diameter. It may be less than 3.2 mm, less than 2.8 mm, or less than 1.8 mm, which are other common endoscope instrument channel for smaller scopes.

The preferred shortest longitudinal length of the outer electrode when fully compressed is in the range of 0.5 mm to 20 mm.

Advantageously, the inner electrode can penetrate as far as required into tissue to be treated, whereas the outer electrode can surround the inner electrode, but be isolated from it by the sheath 11, and electroporation may be performed with desired voltages and other electrical parameters between the electrodes 2 and 3.

As is described below the probe may have different configurations to provide an inner electrode and an outer electrode. In most cases the inner electrode is aligned with the longitudinal axis of the rod or catheter and the second electrode contacts tissue at a location radially spaced apart from the axis and in many cases contacts over an annular area around the axis, and proximally of the distal end of the inner electrode.

Referring to FIG. 2 there is shown a probe 21 before deployment, with a low profile being very small in the lateral/radial direction. In this case an outer electrode 23 has a narrow cylindrical shape before deployment, and there is a needle-shaped inner electrode 25 distally of the outer electrode. There is a proximal rod 24 which contains the conductors linked to the electrodes, and an insulating sheath 22 distally of the outer electrode 23. FIG. 3 shows it with axial compression of the expanding body 23 to illustrate the shape of the expanding body in its expanded state, this is its shape when not compressed on the distal side, by tissue with which it is interacting, as it would be in use.

In the probes 1 and 21 the outer electrode has a pre-deployment diameter greater than that of the rod proximally of it. However, it is envisaged that in other examples the outer electrode, and potentially also the inner electrode, are housed within a sheath pre-deployment, and they are exposed for use by retraction of the sheath.

FIG. 4 shows a probe 50 with an alternative outer electrode expanding body 53 which has a shape designed to potentially confer a greater surface area of contact between the outer electrode expanding body and the tissue against which it is pushed. The distal end 55 is close to radial, conforming to within +/−30° a plane which is normal to the longitudinal axis defined by the elongate support 54. In general, it is preferred that the expanded and unconstrained configuration of the expanding body has a large angle to normal at the distal end. The inner electrode is indicated by the numeral 56 and is needle-shaped.

It will be appreciated that the expanding body may be configured to deform in any of a number of ways to provide effective electrode contact with the proximally facing tissue surface.

FIG. 5 shows a probe 100 with an outer electrode expanding body 103, a needle-shaped inner electrode 110, an insulating sheath 111 around the inner electrode 110 proximally of its tip, and a proximal support rod which houses electrode conductors and is linked to a user actuator. The expanding body 103 has a series of shape memory alloy wire loops which are arranged circumferentially similar to the petals of a flower, and which are heat set to have the shape of an open flower when released from an overlayed sheath. Again, the configuration at the distal end of the expanding body will be close to normal when pressed against a surface around the needle 110, defining a much greater angle to the longitudinal axis than the proximal end 105.

The wire of the outer electrode 103 may be constructed of Nickel Titanium Alloy NiTi and shape set to be in their heat set form at a temperature just below human body temperature, thus ensuring that the NiTi demonstrates its super elastic capabilities and thus the petals are capable of being sheathed and unsheathed repeatably without undergoing plastic deformation and thus maintaining their intended deployed shape. An advantage of this “Petal” design is that the clinician has additional scope for advancing the device further into the tissue which would, when correctly shaped, cause more of the wire loops to come into contact with the tumour surface thus enhancing the contact surface area. In one implementation this could be so implemented as to allow the user vary the diameter of the contact area also.

FIG. 6 shows alternative inner electrode needle configurations, some with radial pores for flow of a treatment substance such as foam, as follows:
110, used in the probe 100, solid with a tip, not acting as a conduit for treatment liquid.
120, hollow with an opening 121 at its distal tip offset to one-side to prevent “tissue-coring” while allowing a single conduit for treatment liquid.
130, hollow with a tip, with radial delivery holes proximally of the tip.
140, hollow with a tip, with radial elongate slots proximally of the tip and having a longer dimension in the longitudinal direction than in the circumferential direction.
150, hollow with a tip, with radial holes at a higher density than in the probe 130. In various embodiments there may be different hole size or density to suit the fluid flow requirements. For example, the apertures may be smaller for lower viscosity liquids.

FIG. 7 shows an alternative probe, 200, in this case with an outer electrode comprising an array of disc-shaped electrodes 202 on a supporting expanding balloon body 201. There is a leading distal needle 210 forming the inner electrode, the proximal end of which is surrounded by an insulating sheath 211. This is one manner of providing that the expanding body is particularly suited for optimum contact with the proximal-facing surface of tissue being treated. Where the outer electrode comprises multiple electrode elements the drive provides a voltage in pulses between the inner electrode 210 and all of the outer electrode elements 202, the latter all being electrically connected together. The electrode elements may be on an expanding mesh of insulated wire.

FIG. 8 shows a further alternative probe, 300, in this case with a mesh 302 of electrode wires on the distal end of a balloon expanding body 301, and a needle 310 and proximal sheath 311.

FIG. 9 shows a further alternative probe, 400, in which the outer electrode comprises a number of separate wire loops 403. The loops 403 are circumferentially arranged around the longitudinal axis, formed from a laser cut shape memory alloy tube or assembly of shape memory wires, which are configured to extend radially inwardly and proximally at the distal end. There is a needle 410 and proximal sheath 411. A benefit of this arrangement is to achieve a lower collapsed profile for delivery through a small endoscope channel.

FIG. 10 is a diagrammatic side view of an alternative probe, 500, in this case with an off-centre, asymmetric, expanding body 501 which may be better suited for full tissue engagement when probes of this type are inserted into tissue at an angle due to geometric obstructions which would prevent their use perpendicular to the tissue to be treated.

FIGS. 11, 12, and 13 show alternative probes with directionality in articulation of the support rod. FIG. 11, shows a probe 600 with a steerable endoscope elongate support 601, and an outer electrode 602 which extends distally and radially when employed for optimum contact with the tissue proximally facing surface. There is a needle 603 providing the inner electrode.

FIG. 12, shows a probe 710 with a steerable endoscope 701, and an outer electrode 702 which extends distally and radially when employed for optimum contact with the tissue proximally facing surface. There is a hinged needle 714 providing the inner electrode which can be articulated about a joint 715 by the device user, the direction of movement of control cables being indicated by the arrows 716. This hinged articulation may be achieved by using a mechanism known for other types of minimally invasive devices including but not limited to push and pull wire control for articulation and patterned laser cutting of the tube to induce the intended bend capability. FIG. 12 also shows a probe 750, in this case having a dish-shaped outer electrode 752 and an elongate and bent inner electrode 751 to allow access to a particular location within tissue. The rod which supports the electrodes is in use steered by the clinician so that the inner electrode penetrates the tissue in the desired direction. Where the outer electrode is dish shaped, as the electrodes 702 and 752 are expandable, they are preferably of shape memory material.

FIG. 13 shows a probe 800 within a steerable endoscope 801, and a dish-shaped outer electrode 802 which extends distally and radially when employed for optimum contact with the tissue proximally facing surface, and a needle 804. The head formed by the electrodes 802 and 804 is hinged to the device's elongate body to allow versatile manipulation.

FIG. 14 shows a probe 900 with an elongate support 901, an outer electrode formed by non-meshing wires 902, and a needle 903 with apertures along its length providing the inner electrode.

FIG. 15 shows a probe 1000 with an elongate support 1001, and an outer electrode 1002 formed by non-meshing wires 1004 supporting flexible wires extending circumferentially, and a needle with apertures along its length providing the inner electrode.

FIGS. 16 and 17 show a probe 1100 with an elongate support 1101 and an outer electrode formed by non-meshing wires 1102. In this case an inner electrode 1103 comprises an elongate rod 1104 terminating at its distal end with a frusto-conical shaped element 1103 which does not pierce tissue but rather presses against it. There is placement of the inner electrode 1103 against the tissue to be treated with the force as required to ensure good electrode-tissue contact.

FIGS. 18(a) and (b) illustrate two states of a probe 1200 of the invention in which the inner electrode is expandable and can expand to a lesser extent than the outer electrode. There is a rod 1202 proximally of the electrodes, and this is preferably steerable. The outer electrode 1201 is an expanding mesh of conductive wires for engaging tissue in an annular region around the longitudinal axis. The inner electrode 1204 may/may not have a needle tip for penetrating tissue to a limited extent. The inner electrode is an expanding mesh to increase the surface area contact with the tissue without tissue penetration within the annular region defined by the distally facing part of the outer electrode 1201. Benefits of this arrangement are to provide a sufficient electrical field by maximising tissue contact area whilst avoiding tissue penetration.

Referring to FIGS. 19(a) to (f) a probe 1250 has a support 1252 with an outer electrode 1251 having an expanding mesh which is dish-shaped when expanded to present an annular mesh to facing tissue. Distally of an insulating sleeve 1253 there is an inner electrode comprising an expanding mesh which is smaller in radial dimension than the outer electrode 1251 and is generally ball shaped. As shown in FIGS. 19(c) and (d) the inner electrode 1254, in use, presses tissue distally to a great er extent than the outer electrode 1251, providing a dish-shaped treatment region 1270. FIGS. 19(e) and (f) show this probe in the form of a prototype pressed against a hard surface for illustrative purposes.

Where the inner electrode comprises an expanding mesh, it may have any of the features described above for the outer electrode, though having a smaller radial dimension. For example, it may have discrete electrode elements mounted to a wire mesh or it may have non-contacting wire loops in a petal-shaped arrangement.

FIGS. 20 and 21 illustrate variations of elements of the probe of this invention which facilitate secure temporary attachment of the probe to the tissue. Specifically, FIG. 20 shows a probe with a rod 1301 and an expanding mesh outer electrode 1302 with meshed wires 1303 and a distal insulating sleeve 1304. There may be a membrane covering part of the outer electrode to facilitate the use of suction to create a vacuum between the electrode body and the target tissue.

The inner electrode may be any of those illustrated in FIGS. 21 (a) to (e) inclusive, as follows, each operated by a proximal actuator at the proximal end of the elongate support 1301.
FIG. 21(a), a clamping element 1310 with a pair of jaws for closing over tissue on the longitudinal axis. In use this can be used to clamp onto the centre of target tissue ensuring maintenance of position during delivery of pulses.
FIG. 21(b), a screw 1320. In use this can be used, via controlled rotation of the centre element, to grip the centre of target tissue ensuring maintenance of position during delivery of pulses.
FIG. 21(c), a suction cup 1330 to form a vacuum. In use this can be used to grip via suction the centre of target tissue ensuring maintenance of position during delivery of pulses.
FIG. 21(d), a partially shrouded shape set element 1340 which when expanded, increases surface contact area with tissue and facilitates maintenance of position during delivery of pulses via suction applied through the gaps present in its body.
FIG. 21(e), a hook. which when revealed forms a hooking engagement with tissue.

In general terms, the probe of the invention has in many embodiments an elongate support with electrical conductors extending from a proximal end to a distal end, the support supporting at least two electroporation electrodes connected to at least two conductors and being mounted to engage tissue. There is an inner electrode which is substantially on the longitudinal axis, or bent off it to some extent, and in many examples, it is suited to penetrate tissue, or press against tissue in the distal direction to the desired depth, or to grip tissue (FIGS. 20 and 21). There is also an outer electrode configured to contact, radially of the longitudinal axis, the proximally facing surface of the tissue being treated. The benefits which arise from this arrangement are selective focal treatment of target tissue which can be delivered through the working channel of an endoscope to align with the workflow of the endoscopy theatre without the requirement for surgery, minimally invasive or otherwise.

In one embodiment, as shown in FIGS. 11 to 13, an electrode is able to track through (or beside) an endoscope when the scope is in retroflexion. The inner and outer electrodes are preferably capable of being deployed and retrieved even when the endoscope is in retroflexion or any position between straight and full retroflexion. The needle depth is preferably adjustable. The outer electrode diameter is preferably adjustable. There is preferably a mechanism in a handle at the proximal end of the elongate support which provides fine user control over the depth to which the The needle is preferably, for some applications, capable of being steered/positioned with respect to the outer electrode and/or the endoscope. This could be achieved through a steerable needle, or a steerable sheath positioned between the needle and the outer electrode. The needle (inner electrode) and outer electrode as a system are preferably capable of being steered/positioned in relation to the endoscope for some applications.

In some embodiments, the needle is hollow and communicating to the handle (proximal end) to facilitate delivery of substances for treatment or management of electrical conductivity. A hollow needle with capped/solid end and side slots/holes facilitates delivery of substances along full length of needle in tissue.

In one embodiment the tip of the inner electrode is flat and does not pierce tissue, so the clinically relevant electric field is near the surface (for example less than 8 mm deep) of tissue.

In one embodiment the outer electrode has multiple separate electrodes that can be activated independently to communicate with the inner electrode or each other.

In general, it is preferred that, whether the inner electrode is configured to penetrate or merely push against tissue, it has a maximum lateral/radial dimension which is 1% to 80%, preferably 20% to 50%, for example 30% of that of the outer electrode. For example, the inner electrode may have an inner diameter of 5 mm and the outer electrode a diameter of 15 mm, leaving a 5 mm radial electrode spacing all around.

The ratio of contact surface area between the outer electrode and the inner electrode can be varied in order to concentrate the electric field strength around the inner electrode. If the outer electrode's contact surface area is >3 times that of the inner electrode the outer electrode can behave as what is, in the state of the art for electrosurgery, referred to as a dispersive pad. The larger this ratio is the greater the similarity between the outer electrode's function and a dispersive pad.

Due to the relatively larger contact area of the outer electrode the current density and thus electric field present in the tissue is significantly lower than that surrounding the inner electrode and thus, with selection of pulse amplitude in combination with a corresponding ratio of outer electrode contact area to inner electrode contact area the location and volume of tissue which achieves clinically significant electric field strength can be localised to just that area which surrounds the inner electrode. A potential advantage of this is that for a relatively low input voltage a large electric field strength differential can be achieved in this locally targeted area. This use of the outer electrode as something akin to a dispersive pad is advantageous in that dispersive pads are typically external to the body and very large, >100 cm³ area, or need to be introduced separately to the electrode being used for treatment and depending on the procedure will thus increase complexity as well as potentially increasing the number of incisions required. The invention has advantages in minimally invasive surgery in that the dispersive pad is collapsible, expandable and does not need an additional entry point to the tissue. Since the grounding pad is relatively close to the site of treatment it avoids some of the downsides of traditional dispersive pads wherein if the pad is too far from the site or has insufficient contact with the patient the current introduced can route to ground elsewhere potentially causing damage to other tissues, nerves, additional pain or burns.

### Advantages

The invention provides an electroporation probe that is low profile on delivery, can control the depth and breadth of the electric field, is easy to navigate in conjunction with the endoscope and applies the PEF in line with the endoscope to ensure the camera can provide adequate visualisation of the tumour. In addition, the density of the electrical field generated is greater than if an alternative solution with multiple needle(s) were used resulting in improved outcomes and response rates. The invention allows for a wider surface area to be treated via the outer electrode in one application with the depth of the electrical field controlled by the surface area of the non-penetrating distal electrode, or depth of the needle penetration which would be typically perpendicular to the mesh.

The above-described outer electrodes allow delivery of a large amount of energy to the tissue during each delivery of a set of electroporation pulses, which means the probe can electroporate relatively large volumes of tissue when compared to individual electrodes. Its pairing with another smaller expanding body or a needle electrode directly within the tissue to be treated means this energy is being forced to pass through the intended target tissue giving the overall probe a good combination of power and precision. In addition, the expanded basket is capable of being made very low-profile, allowing access to the location through typical endoscopes instrument channels before it is expanded to much larger treatment diameters.

In various examples we have described the inner electrode as being a needle which also acts as a conduit, possibly with radial apertures. In another example the needle is closed at its distal tip and the apertures in its wall are the only paths for flow of a fluid. The term “conductor” means one or more conductive wires which extend along or through the elongate support and are linked with the inner and outer electrodes.

In various examples the inner electrode has the capability of expanding to a lesser extent than the outer electrodes, improving the field uniformity between the outer electrode and the inner electrode.

Components of embodiments can be employed in other embodiments in a manner as would be understood by a person of ordinary skill in the art. The invention is not limited to the embodiments described but may be varied in construction and detail.

1. An electroporation probe comprising:
an elongate support with conductors, said elongate support supporting:
an inner electrode linked with at least one of said conductors and being configured to engage tissue, the inner electrode having an inner electrode axis, and
an outer electrode linked with at least one of said conductors and being configured to engage tissue radially outwardly and proximally of the inner electrode.

2. The electroporation probe as claimed in claim 1, wherein the inner electrode comprises a needle configured to penetrate tissue. needle.

3. The electroporation probe as claimed in claim 1, wherein the outer electrode is configured to contact tissue over an annular area around the axis of the inner electrode and proximally of the distal end of the inner electrode.

4. The electroporation probe as claimed in claim 1, wherein the inner electrode is mounted along a longitudinal axis of the elongate support; and wherein the inner electrode and the outer electrode are collapsed in the elongate support and are expandable when the elongate support is retracted.

5. The electroporation probe as claimed in claim 1, wherein the elongate support comprises an endoscope, and the electrodes are mounded in a collapsed state within a working or biopsy channel of the endoscope.

6. The electroporation probe as claimed in claim 1, wherein the inner electrode is hollow for at least some of its length and is configured for delivery of a fluid to tissue being treated, and wherein the needle has radial apertures for radial delivery of a fluid in addition to or instead of through a needle tip.

7. (canceled)

8. The electroporation probe as claimed in claim 1, wherein the outer electrode is deformable for engagement with a proximal facing surface of tissue being treated; and wherein the outer electrode comprises an expanding mesh supporting or being integral with electrode elements.

9. (canceled)

10. (canceled)

11. (canceled)

12. The electroporation probe as claimed in claim 1, wherein the outer electrode is deformable for engagement with a proximal facing surface of tissue being treated; and wherein the outer electrode comprises an expanding body supporting discrete electrode elements at a distal end thereof.

13. (canceled)

14. The electroporation probe as claimed in claim 1, wherein a needle depth is adjustable relative to the elongate support; and wherein a lateral dimension of the outer electrode is adjustable.

15. (canceled)

16. (canceled)

17. The electroporation probe as claimed in claim 1, wherein the outer and/or inner electrodes are steerable in relation to the elongate support.

18. (canceled)

19. The electroporation probe as claimed in claim 1, wherein the inner electrode includes an electrode element which is not needle-shaped but has a lateral dimension in the range of 1 mm to 10 mm to press distally against tissue being treated; and wherein the inner electrode comprises an expanding mesh.

20. (canceled)

21. (canceled)

22. The electroporation probe as claimed in claim 1, wherein the inner electrode is deformable for engagement with a proximal facing surface of tissue being treated; and wherein the inner electrode comprises an expanding body supporting electrode elements.

23. (canceled)

24. (canceled)

25. (canceled)

26. The electroporation probe as claimed in claim 1, wherein the outer electrode includes a suction element part to create a vacuum between the outer electrode and target tissue.

27. The electroporation probe as claimed in claim 1, wherein the inner electrode includes at least one selected from:
an element for gripping tissue,
an expandable body having a maximum lateral dimension less than that of the outer electrode,
wire loops which do not connect to each other when relaxed.

28. (canceled)

29. (canceled)

30. (canceled)

31. The electroporation probe as claimed in claim 1 wherein, in a probe relaxed and unconstrained state, the inner electrode has a lateral dimension which is 20% to 50% of the lateral dimension of the outer electrode.

32. (canceled)

33. An electroporation apparatus comprising an electroporation probe and a pulse generator, wherein the electroporation probe comprises an elongate support with conductors, said elongate support supporting an inner electrode linked with at least one of said conductors and being configured to engage tissue, the inner electrode having an inner electrode axis, and an outer electrode linked with at least one of said conductors and being configured to engage tissue radially outwardly and proximally of the inner electrode, and wherein said pulse generator is linked with said conductors for delivery of pulses to the electrodes.

34. The apparatus as claimed in claim 33, wherein the inner electrode is hollow for at least some of its length and is configured for delivery of a fluid to tissue being treated, and the apparatus further comprises a pump for delivery of a fluid through the inner electrode.

35. (canceled)

36. A method of treatment of tissue in a human or animal body, the method being performed with an electroporation apparatus comprising an electroporation probe and a pulse generator, wherein the electroporation probe comprises an elongate support with conductors, said elongate support supporting an inner electrode linked with at least one of said conductors and being configured to engage tissue, the inner electrode having an inner electrode axis, and an outer electrode linked with at least one of said conductors and being configured to engage tissue radially outwardly and proximally of the inner electrode, and wherein said pulse generator is linked with said conductors for delivery of pulses to the electrodes; and wherein the method comprises:
deploying a probe of the electroporation apparatus within the body so that the inner electrode and the outer electrode contact tissue with the outer electrode radially outside of the inner electrode and spaced apart from the inner electrode and applying voltage pulses to the electrodes for electroporation treatment of the tissue between and adjacent said electrodes.

37. The method as claimed in claim 36, wherein the inner electrode is moved longitudinally to provide a desired action of pressing against or penetrating the tissue; and wherein the inner electrode is moved longitudinally relative to the outer electrode to achieve a desired differential in longitudinal positions of said electrodes.

38. (canceled)

39. (canceled)
